(19) United States
(12) Patent Application Publication
(10) Pub. No.: US 2006/0235449 A1
(43) Pub. Date: Oct. 19, 2006

(54) Vascular intimal lining removal assembly

(75) Inventors: Peter J. Schubart, Los Altos Hills, CA (US); Gordon Kavanaugh Stokes, Norfolk, VA (US); D. Bruce Modesitt, San Carlos, CA (US); Ted S. Thorson, Sunnyvale, CA (US)

Correspondence Address: HAYNES BEFFEL & WOLFELD LLP, P O BOX 366, HALF MOON BAY, CA, 94019, US

(73) Assignee: Vascular Architects, Inc., a Delaware Corporation, San Jose, CA (US)

(21) Appl. No.: 11/109,450

(22) Filed: Apr. 19, 2005

(51) Int. Cl. A61B 17/22

(52) U.S. Cl. 606/159

(57) ABSTRACT

A vascular lining removal assembly comprises a plurality of slats, each having inner and outer surfaces, configured to pass along a vascular cleavage plane to removingly engage a length of vascular lining. In some embodiments the assembly may also include means for engaging the length of vascular lining to aid removal of the length of vascular lining. The inner surface of at least one of the slats may comprise an inwardly opening passageway to permit direct access to the length of vascular lining by a tool.

### CROSS-REFERENCE TO OTHER APPLICATIONS

None.

### FEDERALLY SPONSORED RESEARCH OR DEVELOPMENT

None.

### BACKGROUND OF THE INVENTION

Remote endarterectomy is a procedure by which an incision is made at a position along a blood vessel to provide access to the blood vessel by some type of ring stripper. The remote endarterectomy procedure is typically accomplished when the blood vessel is partially or totally occluded by plaque. The ring stripper, which typically consists of a ring at the end of a long thin handle, is typically passed along a subintimal vascular cleavage plane between layers of the blood vessel. The vascular cleavage plane is typically between the tunica intima (also called the intimal layer) and the tunica media or between the tunica media and the tunica adventitia. The procedure is typically conducted under fluoroscopic guidance thus dissecting the intimal layer and the occlusion from the remainder of the vessel wall to a position distal of the occlusion. The ring stripper is then removed and a remote cutter can be used to transect the separated intimal layer distally of the occlusion. This permits the intimal layer and the occlusion to then be removed. A severing ring stripper, such as shown in U.S. Pat. Nos. 6,328,749 and 5,843,102, may be used to eliminate the need for a separate ring stripper and a separate remote cutter.

One or more natural cleavage planes typically exist along a blood vessel. However, in some places the natural cleavage plane may be interrupted, such as by a highly adhesive plaque deposit or by a calcified buildup. Such an interruption effectively prevents the passage of a conventional dissection device past the interruption to at least impede or temporarily halt the procedure.

Instead of using a ring stripper, other types of tissue dissectors can be used along vascular cleavage planes to cleave plaque from the remainder of the blood vessel. One example of such a dissector is disclosed U.S. Pat. No. 6,506,178. The dissector is typically inserted subintimally in approximately three quadrants around the circumference of the plaque. The final quadrant may then be cleaved with a conventional ring stripper. If the plaque needs to be cut to free the distal end point, a severing ring stripper as disclosed in U.S. Pat. No. 6,328,749 is typically used.

Sometimes the distal end point thins or feathers to relatively normal intimae, in which case, the plaque can be freed by avulsion with just a conventional ring stripper. In any case, once the cleavage plane has been developed and the distal end point is freed or terminated, the plaque must be removed. Removal of the plaque or other occlusion is often tedious and difficult. It is complicated by the fact that plaque is often discontinuous and fragile. Even a slight tensioning during extraction will often cause the plaque to snap midway leaving a distal section of plaque in the artery. Attempts to push the plaque out from the distal end often results in the plaque bunching up or train wrecking in the vessel.

### BRIEF SUMMARY OF THE INVENTION

The present invention addresses some of the problems that arise when cleaning plaque from the vessel wall and when removing the severed intimal layer and occlusion during an endarterectomy procedure.

A first aspect of the present invention is directed to a vascular lining removal assembly comprising a plurality of slats, each having inner and outer surfaces, configured to pass along a vascular cleavage plane to removingly engage a length of vascular lining. In some embodiments the assembly may also include means for engaging the length of vascular lining to aid removal of the length of vascular lining.

A second aspect of the invention is directed to a vascular lining removal element comprising a slat, having inner and outer surfaces, configured to pass along a vascular cleavage plane to removingly engage a length of vascular lining. The slat has a distal portion having an average thickness T, an average width W and a length L, with W being at least 3 times T and L being at least 80 times W. In some embodiments, where the target length of vascular lining to be removed is TL, L is 50% to 500% as long as TL while in other embodiments L is at least 150% as long as TL.

A third aspect of the invention is directed to a method for removing a length of vascular lining from a blood vessel. The method includes positioning a plurality of slats, each having inner and outer surfaces, along a cleavage plane of a blood vessel; removingly engaging a length of vascular lining by the slats; and removing the slats and a removed length of vascular lining therewith from the remainder of the blood vessel, the removed a length of vascular lining having a length RL. According some embodiment of the invention, the removingly engaging and removing steps are carried out without any user-applied constricting force on the slats. According to the other embodiments of the invention, the removingly engaging step comprises constricting the length of vascular lining by the slats.

A fourth aspect of the invention is directed to a vascular lining removal assembly comprising a plurality of slats, each having inner and outer surfaces, configured to pass along a vascular cleavage plane to removingly engage a length of vascular lining. The inner surface of at least one of the slats comprises a passageway extending along at least a portion of the inner surface, at least a portion of the passageway opening inwardly to permit direct access to the length of vascular lining by a tool. In some embodiments a radially inwardly extendable tool is carried by at least one slat, the tool being movable between a first, radially retracted state and a second, radially inwardly extended state so to engage the length of vascular lining in the second state. The tool may comprise, for example, at least one of a cutter, a tissue separating tool and a lumen re-entry tool.

A fifth aspect of the invention is directed to vascular lining treatment assembly. The assembly comprises a slat, having inner and outer surfaces, configured to pass along a vascular cleavage plane to engage a length of vascular lining. The inner surface of the slat comprises a passageway extending along at least a portion of the inner surface. At least a portion of the passageway opens inwardly to permit direct access to the length of vascular lining. A tool engages and passes along the passageway of the slat to permit engagement of the length of vascular lining by the tool.

A sixth aspect of the invention is directed to a vascular lining removal element comprising a slat, having inner and outer surfaces, configured to pass along a vascular cleavage plane to removingly engage a length of vascular lining. The inner surface comprises a passageway extending along at least a substantial portion of the inner surface. At least a portion of the passageway opens inwardly to permit direct access to the length of vascular lining by a tool. The slat has a distal portion with an average thickness T, an average width W and a length L, with W being at least 3 times T and L being at least 80 times W.

A seventh aspect of the invention is directed to a method for removing a length of vascular lining from a blood vessel. A plurality of slats, each having inner and outer surfaces, is positioned along a cleavage plane of a blood vessel, the inner surface comprising a tool passageway extending along at least a portion of the inner surface, at least a portion of the tool passageway opening inwardly to permit direct access to a length of vascular lining by a tool. The length of vascular lining is engaged through at least one tool passageway by a tool. The length of vascular lining is removably engaged by the slats. The slats and the length of vascular lining therewith are removed from the remainder of the blood vessel. The tool is removed from the blood vessel. The slats are removed from the blood vessel.

Various features and advantages of the invention will appear from the following description in which the preferred embodiments have been set forth in detail in conjunction with the accompanying drawings.

### BRIEF DESCRIPTION OF THE DRAWINGS

FIG. 1 is an isometric view of a vascular lining removal assembly made according to the invention;

FIG. 2 is an enlarged isometric view of one of the slats of the assembly in FIG. 1;

FIG. 3 is an end of view of the distal end of the slat of FIG. 2;

FIG. 4 is a simplified cross-sectional view showing the distal portion of the assembly of FIG. 1 within a blood vessel with the slats passing along a cleavage plane between the intimae and media to surround a target length of vascular lining;

FIG. 5 is a simplified cross-sectional view of a blood vessel containing an occlusion;

FIG. 6 illustrates the assembly of FIG. 1 with a filament wrapped around the assembly to permit the assembly to be constructed about a target length of vascular lining;

FIG. 7 is an alternative embodiment of the slat of FIG. 2 including a guide track on the outer surface, the guide track used to help guide a pusher element;

FIGS. 8 and 9 illustrate the inner surface of a slat having an inner track, the slat housing a removable guide wire guide;

FIG. 10 illustrates a pair of slats having edges configured to be engaged by the enlarged edges of a slat edge coupler;

FIG. 11 is a cross-sectional view of a blood vessel having a vascular lining removal assembly including the slats and coupler of FIG. 10 positioned along a cleavage plane with the slats constricting the target length of vascular lining by virtue of the edges of the slats being joined by slat edge couplers;

FIG. 12 is a partial cross-sectional view of a slat having one-way friction elements along the inner surface to help prevent undesirable slippage between the slats and length of vascular lining during the removal process;

FIG. 13 is a cross-sectional view of a further embodiment of a slat having vacuum ports connecting a vacuum manifold to the inner surface of the slat to help prevent undesirable slippage between the slats and length of vascular lining during the removal process;

FIGS. 14 and 15 are partial cross-sectional views of a still further embodiment of a slat in which the slat has an open interior housing an inflatable bladder, the bladder having friction elements housed within openings formed in the inner surface of the slat so that when the bladder is inflated from the uninflated condition of FIG. 14 to the inflated condition of FIG. 15, the friction elements extend radially inwardly to engage target tissue to help prevent undesirable slippage between the slats and length of vascular lining during the removal process;

FIGS. 16 and 17 are end and cross-sectional views of an embodiment of a slat in which the slat has a high friction inner surface initially covered by a shield, the shield being removable after the slat is in position to permit the high friction surface to engage the vascular lining to help prevent undesirable slippage between the slats and length of vascular lining during the removal process;

FIG. 18 is an isometric view of an embodiment of a slat having an inner track along its inner surface;

FIGS. 19 and 20 are isometric and cross-sectional views of a slat having a radially extendable tool along an inner portion of the slat, the tool shown in a radially contracted state;

FIGS. 21 and 22 illustrate the structure of FIGS. 19 and 20 with the tool in a radially extended condition so to radially constrict a target length of vascular lining to help prevent undesirable slippage between the slats and length of vascular lining during the removal process;

FIG. 23 illustrates a slat having both an outer guide track and an inner track by:

FIGS. 24 and 25 are forceps types of tools which can be guided along the inner track of the slat of FIG. 18 and adapted to engage a target length of the vascular lining;

FIGS. 26 and 27 illustrate the distal ends of cutting tools carried by and extending from the distal end of a slat having a concave cutting edge and a convex cutting end;

FIG. 28 illustrates the distal end of a scissors-type cutter carried by and extending from the distal end of a slat, the scissors-type cutter configured to cut when closed or opened, or both;

FIGS. 29 and 30 illustrates a clamshell type of tissue separator tool carried by and extending from the distal end of a slat, the tool shown in a closed state;

FIGS. 31 and 32 illustrate the tool of FIGS. 29 and 30 in a tissue separating opened state;

FIG. 33 shows a circular blade type of cutting tool carried by and extending from the distal end of a slat, the blade being a rotating and/or reciprocating blade;

FIG. 34 shows a lumen re-entry tool, including an inwardly angled exit lumen and a hollow needle extending from the exit lumen, carried by and extending from the distal end of a slat;

FIG. 35 shows the structure of FIG. 34 with the re-entry tool positioned along the inner track of the slat with the hollow needle passing through the inner track;

FIG. 36 illustrates a distally oriented cuplike cutter type of tool;

FIG. 37 illustrates the distally oriented cuplike cutter tool of FIG. 36 extending from the inner track of a slat, the cutter being movable distally to engage and remove material;

FIG. 38 illustrates a proximally oriented cuplike cutter type of tool;

FIG. 39 illustrates the proximally oriented cuplike cutter tool of FIG. 38 extending from the inner track of a slat, the cutter being movable proximally to engage and remove material;

FIGS. 40 and 41 are overall and end views of a cutting tool having first and second legs extending along the interior of adjacent slats with a blade secured to and extending between the distal ends of the legs;

FIG. 42 shows a tool similar to that of FIG. 40 but having a notched blade;

FIG. 43 shows a tool similar to that of FIG. 40 but having a rotating and/or reciprocating blade;

FIG. 44 shows a tool similar to that of FIG. 40 but having a chisel tip instead of a blade;

FIG. 45 shows a tool similar to that of FIG. 40 but having a clamshell type of tissue separator instead of a blade;

FIG. 46 shows a tool similar to that of FIG. 40 but having a scissors type of cutter instead of a stationary blade;

FIGS. 47 through 58 disclose various embodiments of the invention in which the slats have keyhole type of openings along their side edges to permit an elongate cutter to be guided along the edge of one slat, see FIGS. 47-52, or between the side edges of the adjacent slats, see FIGS. 53-58;

FIGS. 47 and 48 disclose a further embodiment invention in which the elongate cutter has a blade pivotally mounted at its distal end;

FIGS. 49 and 50 disclose an embodiment similar to that of FIGS. 47 and 48 but with the proximally facing blade in an extended, use state;

FIGS. 51 and 52 illustrate an embodiment similar to that of FIGS. 49 and 50 but with a distally facing blade;

FIGS. 53 and 54 shows further embodiment in which the cutter includes a scissors type of blade assembly;

FIGS. 55 and 56 show an embodiment with a blade extending between the side edges of adjacent slats;

FIGS. 57 and 58 show an embodiment similar to FIGS. 55 and 56 but with a notched blade;

FIG. 59 shows a slat having a porous outer surface opening into an interior passageway of the slat for delivery of an agent; and

FIG. 60 shows an embodiment similar to that of FIG. 59 but in which the outer surface is closed so to be useful for radiation therapy.

### DETAILED DESCRIPTION OF THE INVENTION

The present invention is described with reference to several embodiments with like elements referred to with like reference numerals.

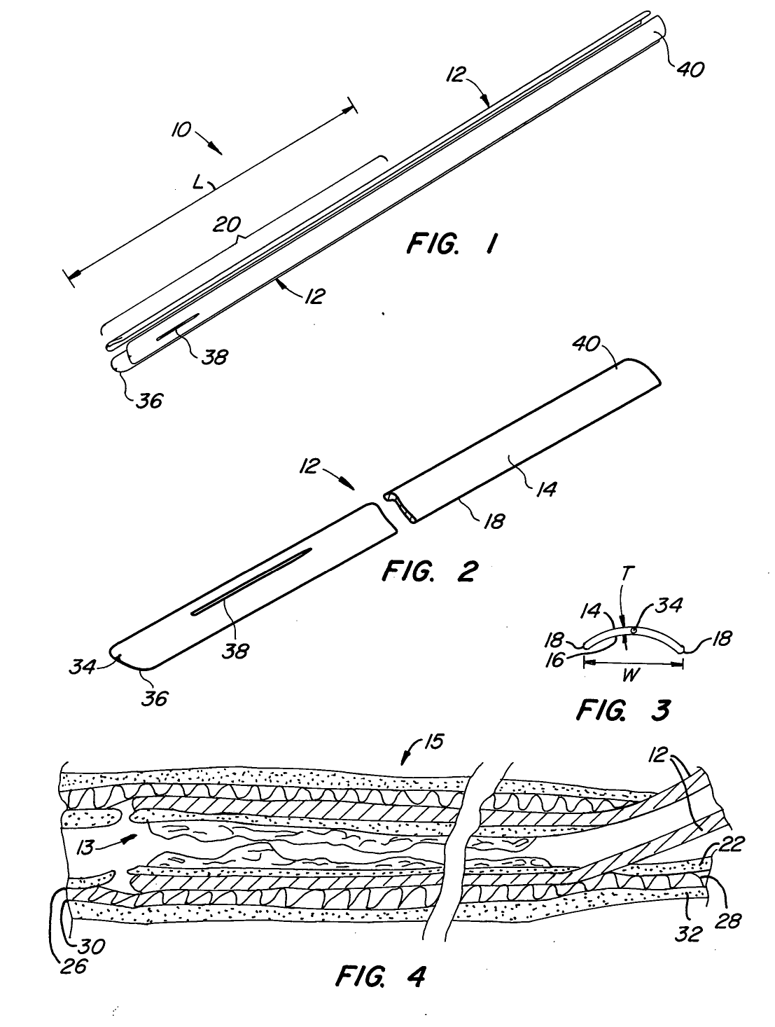

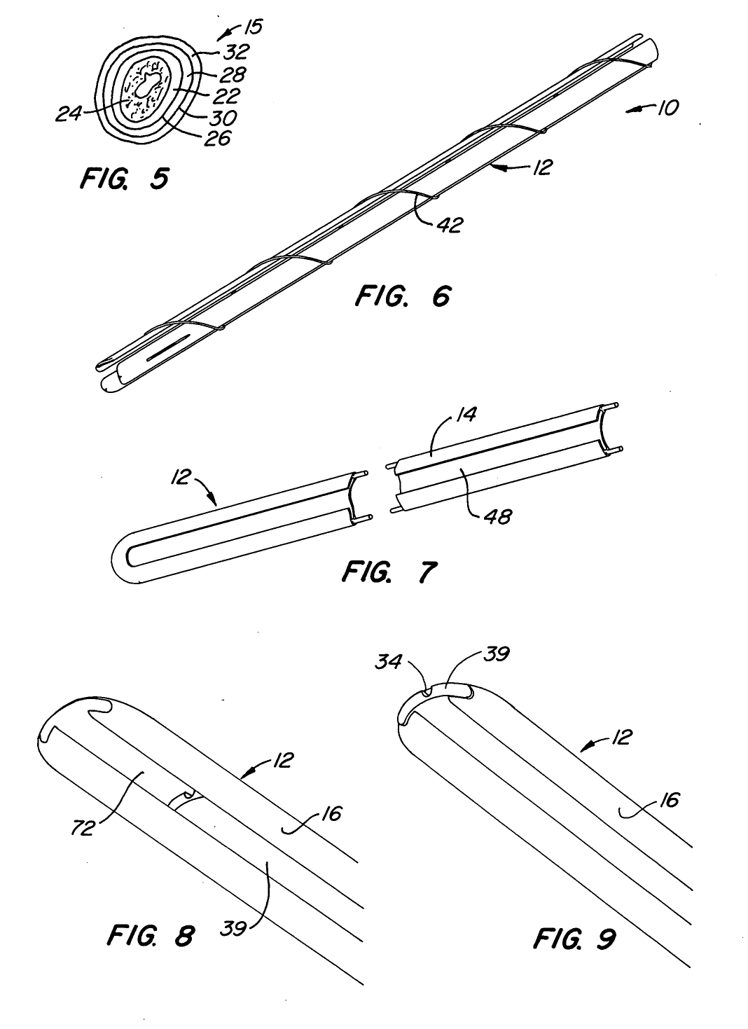

A vascular lining removal assembly 10 is shown in FIG. 1 as comprising three slats 12. Particular situations may call for the use of only two slats 12 or more than three slats 12. Each slat 12 has a distal portion 20 sized and configured to engage a target length 13 of vascular lining within a blood vessel 15, see FIGS. 4 and 5, as discussed in more detail below. Distal portion 20 of slat 12 is shown in FIGS. 2 and 3 as including a convex outer surface 14 and a concave inner surface 16, surfaces 14, 16 joined by two generally parallel edges 18. Edges 18 may be other than parallel, such as tapered. Although in the disclosed embodiment of FIGS. 1-3 slats 12 have the same general cross-sectional shape along their entire lengths, the proximal portions of slats 12, because they are not intended to engage target length 13, may have other cross-sectional shapes.

Target length 13 of the vascular lining typically includes either (1) intima 22 and occlusion 24 when slats 12 are passed along cleavage plane 26 between intima 22 and media 28 (as in FIG. 4), or (2) media 28, intima 22 and occlusion 24 when slats 12 are passed along cleavage plane 30 between media 28 and adventitia 32. (not shown).

Distal portion 20 has a length L, an average width W and average thickness T. Width W is preferably at least 3 times T and L is preferably at least 80 times W. Assuming the target length of vascular lining is TL, L is preferably at least 150% as long as target length TL and is typically 50% to 500% as long as target length TL. In one preferred embodiment, designed for use with a target length 13 of about 10 mm to 500 mm, each slat 12 is made of stainless steel and has a length L of about 400 mm, an average width W of 5 mm and an average thickness T of 1 mm.

Other materials or combinations thereof may be used for slats 12. Examples of such other materials include single or multiple stainless steel alloys, nickel titanium alloys (e.g., Nitinol), cobalt-chrome alloys (e.g., ELGILOY® from Elgin Specialty Metals, Elgin, Ill; CONICHROME® from Carpenter Metals Corp., Wyomissing, Pa.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyester (e.g., DACRON® from E. I. Du Pont de Nemours and Company, Wilmington, Del.), polypropylene, polytetrafluoroethylene (PTFE), expanded PTFE (ePTFE), polyether ether ketone (PEEK), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), polylactic acid (PLA), polydioxanone, and pseudo-polyamino tyrosine-based acids, extruded collagen, silicone, zinc, echogenic, radioactive, radiopaque materials.

Although slats 12 typically are identical, they may have different dimensions. Slats 12 are preferably flexible but possess an appropriate amount of flexural strength for the particular procedure. In some situations the flexibility of slats 12 may vary along their lengths; for example tapered slats may be used, such as in a tapering vessel, which would result in a varying flexibility. Also, the internal material structure of slats 12 may very resulting in a very flexibility. Each slat 12 may have a guide wire lumen 34 extending from the tip 36 of each slat 12 to an exit slot 38 along outer surface 14. This permits slats 12 to be guided along the appropriate cleavage plane 26, 30 by a pre-positioned guide wire, not shown. After slats 12 have been properly positioned with distal portions 20 of slats 12 surrounding and engaging the target length 13, see FIG. 4, the slats engage target length 13 to facilitate removal of the target length of vascular lining. This can be done in a number of ways.

FIGS. 8 and 9 illustrate inner surface 16 of slat 12 having an inner track 72. To permit slat 12 with inner track 72 to be guided by a guide wire, a removable guide wire guide 39 is positioned within the interior of slat during the placement of the slat. After proper placement of slat 12, guide wire guide 39 and the guide wire may be removed from slat 12.

One way of engaging target length 13 is by the physician or other medical personnel grasping the proximal ends 40 of slats 12 and squeezing the proximal ends causing distal portions 20 to constrict target length 13. Another way to constrict target length 13 is by wrapping one or more filaments 42 around slats 12, as shown in FIG. 6, and then pulling on the filaments from the proximal end 40. Another way to constrict target length 13 is through the use of a radially constrictable braid or mesh tubular element, not shown; pulling on such a tubular element causes the tubular element to squeeze slats 12 against and therefore constrict target length 13 as is desired. Placement of filament 42 or a constrictable tubular element can be done after slats 12 are in place surrounding target length 13 of vascular lining with the aid of a constricting element pusher, not shown, to push the constricting element over slats 12. If a pusher is used, it may be desirable to provide one or more of slats 12 with a pusher guide track 48 as shown in FIG. 7.

In some situations target length 13 may be sufficiently constricted by slats by the inward force exerted on slats 12 by the vessel wall. This permits target length 13 to removed without any user-applied or other additional constriction force on the slats.

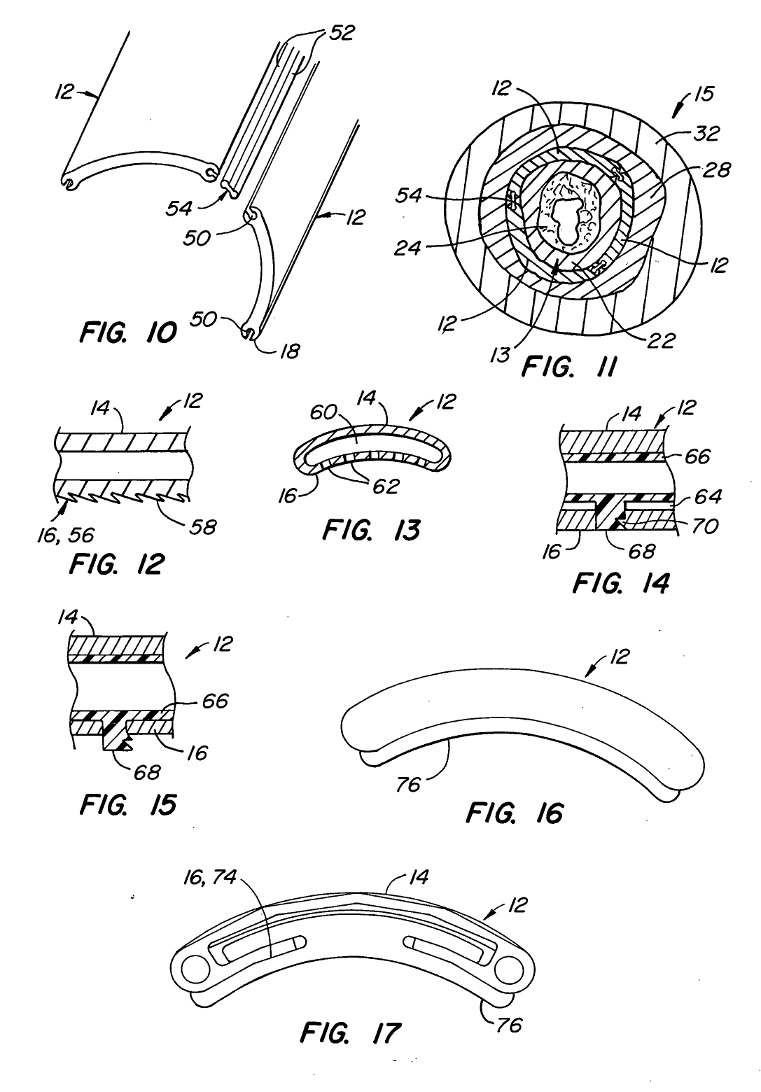

Another way of constricting target length 13 is illustrated in FIGS. 10 and 11. Slats 12 have keyhole type openings 50 extending along their edges 18. Once slats 12 are in position along cleavage plane 26 and/or 30 and surrounding target length 13, edges 18 of adjacent slats 12 at proximal ends 40 are placed adjacent to one another. The enlarged edges 52 of slat edge couplers 54 are then passed into the adjacent openings 50 causing the adjacent edges 18 to be pulled together thus constricting target length 13 of the vascular lining. Coupler 54, which has a fixed width, could be replaced by a coupler having an adjustable width to permit the user to adjust the amount of constriction. One such coupler 54 could be in the form of a ladder and have cylindrical rails connected by rungs, the rungs being hinged to the rails in a parallelogram arrangement to permit the distance between the rails to the changed.

A further way of engaging target length 13 of vascular lining is through the use of enhanced friction elements along inner surface 16 of one or more slats 12. One type of enhanced friction element is a one-way friction element 56 shown in FIG. 12. One-way friction element 56 has numerous proximally oriented elements 58 so that inner surface 16 creates a relatively small frictional force between inner surface 16 and target length 13 during placement of slat 12 as slat 12 moves in a distal direction along a cleavage plane 26 and/or 30. However, during removal of target length 13, elements 58 engage target length 13 of the vascular lining, preferably along the entire length of target length 13, to help remove the target length from the remainder of blood vessel 15. The disclosed elements 58 are substantially rigid; they may also be flexible or pivotally mounted elements.

FIG. 13 illustrates a still further way of engaging target length 13. Slat 12 comprises a vacuum manifold 60 and numerous vacuum ports 62 along inner surface 16. Once slats 12 are properly in position surrounding target length 13, a partial vacuum is formed in manifold 60 causing target length 13 to adhere to inner surface 16.

FIG. 14 is a cross-sectional view of a portion of a slat 12 having an open interior 64, similar to vacuum manifold 60 of FIG. 13, containing a bladder 66. A number of friction elements 68 extend from bladder 66 and pass into openings 70 formed in inner surface 16. FIG. 14 illustrates bladder 66 in a relaxed, uninflated state with friction elements 68 completely or substantially housed within openings 70. After slats 12 are properly positioned surrounding target length 13, bladder 66 is pressurized as shown in FIG. 15 causing friction elements 66 to extend out through openings 70 and engage target length 13 of the vascular lining. Friction elements 68, due to their ability to retract and extend, act as one-way friction elements. The engagement of the tissue of target length 13 helps to secure target length 13 to assembly 10 during removal of the target length.

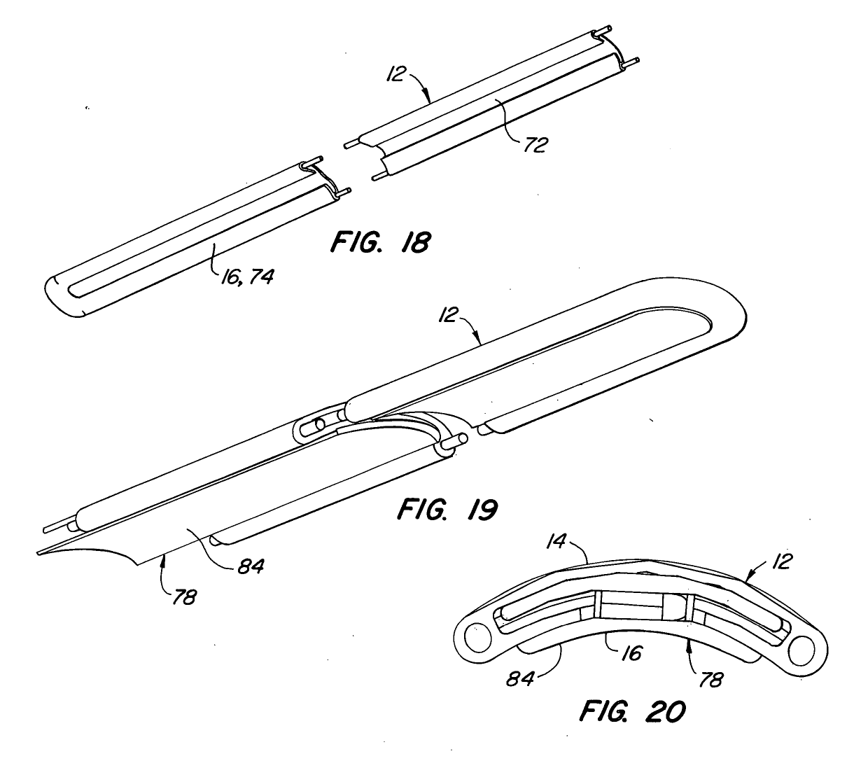

FIGS. 16, 17 and 18 are end, cross-sectional and isometric views of another version of slat 12 designed to engage target length 13. Slat 12 has an inner passageway or track 72 along inner surface 16. Inner surface 16 is a high friction surface 74; see FIGS. 16 and 17, to provide significant frictional engagement between slat 12 and target length 13 of vascular lining during the removal process. However, to accommodate deployment of slats 12, a removable, low friction shield 76 is used to cover high friction surface 74 during the deployment procedure. After proper deployment, shield and 76 is removed, see FIG. 18, typically by pulling on the shield in a proximal direction, to permit high friction surface 74 to properly engage target length 13.

As used herein, friction includes both (1) conventional static and dynamic sliding friction forces, such as from roughened surfaces or molecular level interactions, and (2) sliding-movement-inhibiting friction type force between contacting surfaces created by macro surface interactions, such as a pins or pegs extending into the vascular lining. Friction forces resulting from molecular level interactions may be created using an adhesive or not.

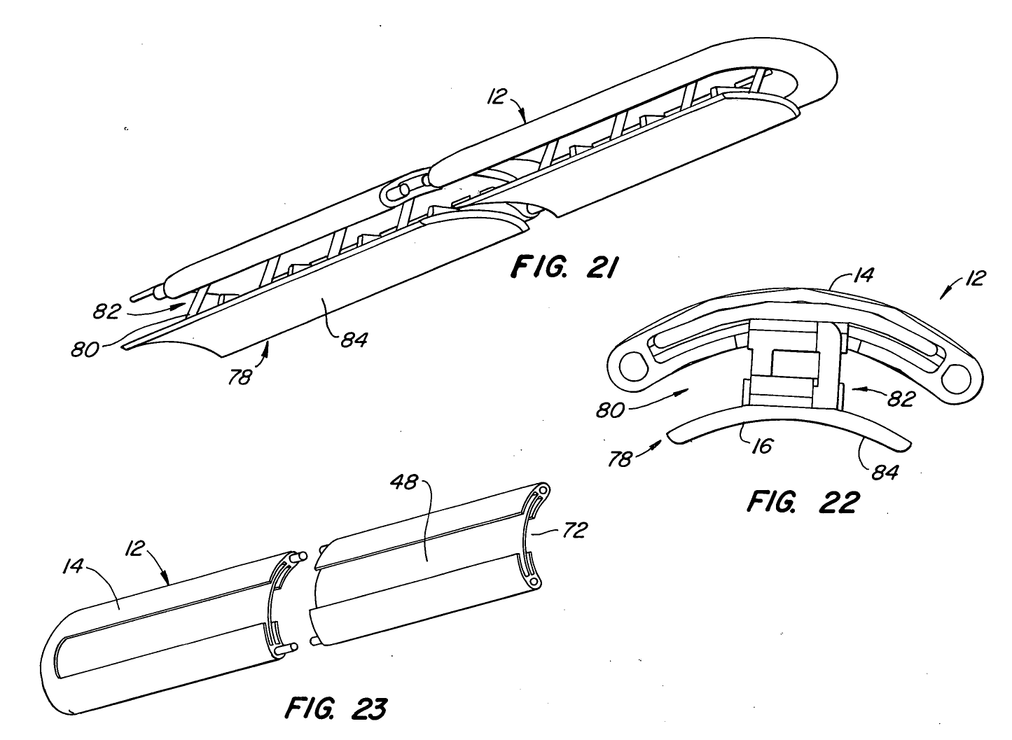

FIGS. 19-22 illustrate a further embodiment in which a slat 12, similar to that shown in FIG. 18, has a radially extendable tool 78 mounted along inner track 72. Tool 78 includes an extension mechanism 80 comprising swing arms 82 that pivotally mount a tissue engaging pad 84 to slat 12 for movement between the radially retracted position of FIGS. 19 and 20 and the radially inwardly extended position of FIGS. 21 and 22. After placing slats 12 in position engaging target length 13 of the vascular lesion with one or more of the slats having tool 78, one or more pads 84 are place in the radially extended position of FIGS. 21 and 22 thus constricting target length 13 of the vascular lining. Doing so facilitates removal of the target length of vascular lining. Other types of the radially extendable tools, such as an inflatable bag, balloon or bellows, could also be used.

FIG. 23 illustrates a further alternative embodiment in which a slat 12 has both an inner track 72, typically for use with a tool used for engaging the vascular lining, and an outer track 48, typically used to guide a pusher. Either inner track 72 or outer track 48 may be used to facilitate mounting a radially extendable tool, such as tool 78.

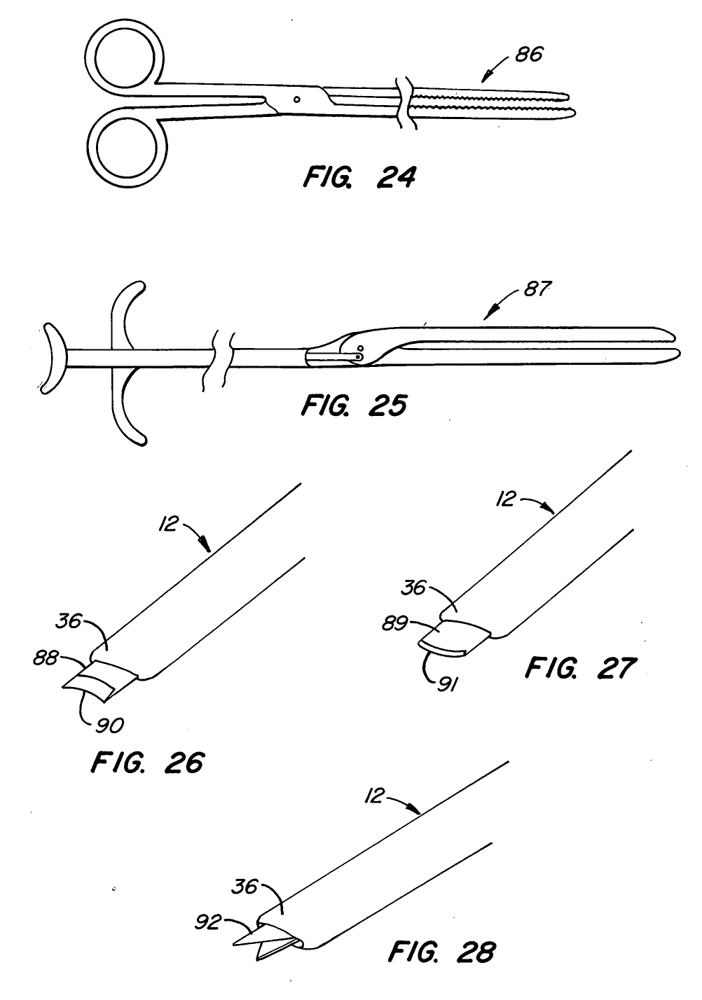

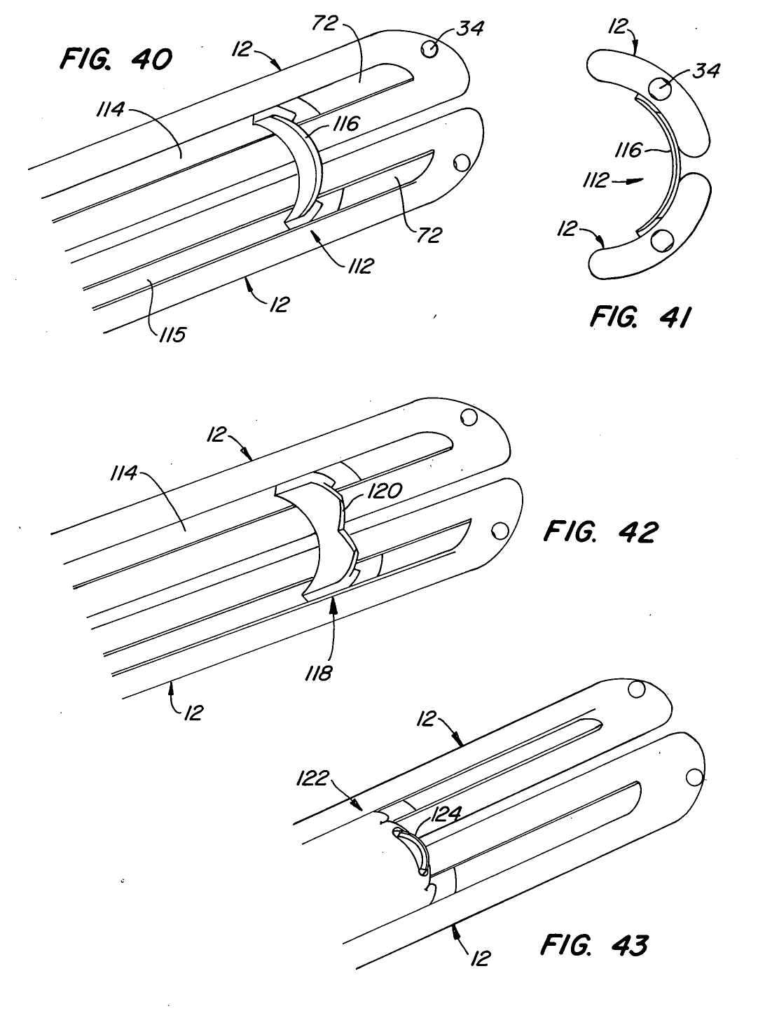

The embodiment of slat 12 of FIG. 18, including inner track 72, can be used with various other tools which are guided along inner track 72 and adapted to engage target length 13 of the vascular lining. Examples of such tools are illustrated in FIGS. 24-39. One or more of these tools may also be usable along outer track 48.

One or more natural cleavage planes typically exist along a blood vessel. However, in some places the natural cleavage plane may be interrupted, such as by a highly adhesive plaque deposit or by a calcified buildup that effectively prevents the passage of a dissection device past the interruption. With the present invention slats 12 can be positioned on either side of the cleavage plane interruption to permit the removal of target length 13 of the vascular lining to proceed with or without the use of various cutting and other tools, such as those disclosed in FIGS. 24-58.

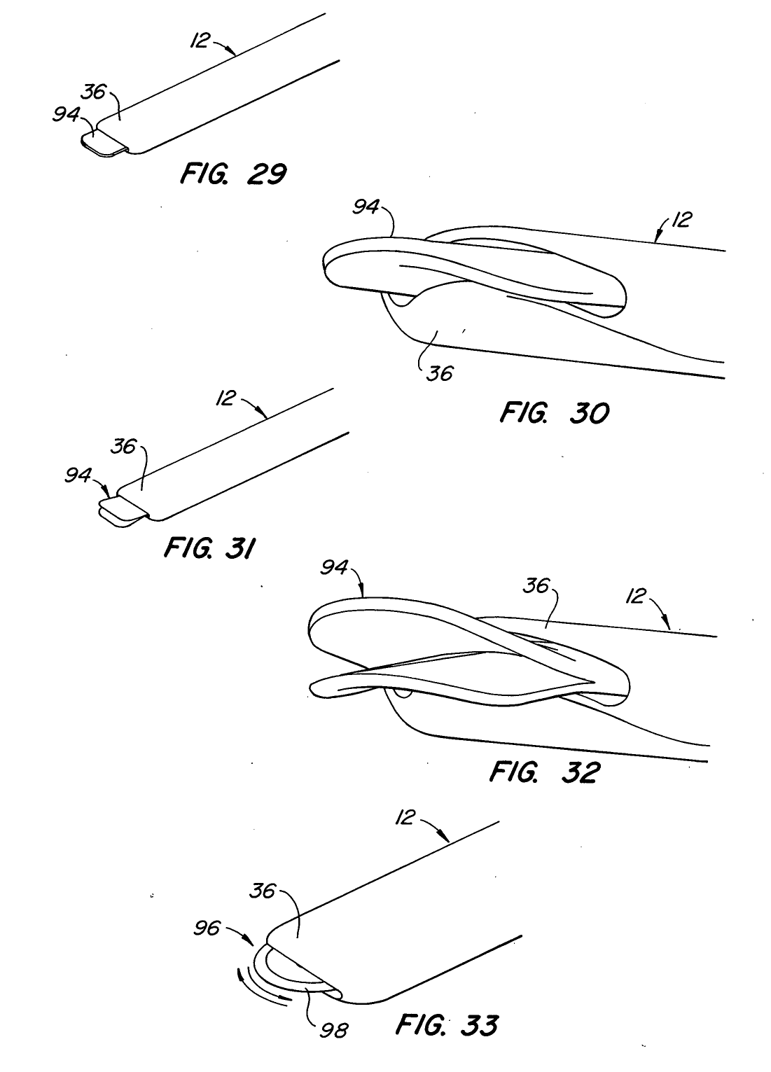

FIGS. 24 and 25 are forceps types of tools 86, 87 which can be guided along inner track 72 of slat 12 of FIG. 18 and engage, for example, a target length of the vascular lining 13. FIGS. 26 and 27 illustrate the distal ends of cutting tools 88, 89 carried by and extending from tip 36 of slat 12. Tools 88, 89 have concave and convex cutting ends 90, 91, respectively. FIG. 28 illustrates the distal end of a scissors-type cutter 92 carried by and extending from tip 36 of slat 12. Scissors-type cutter 92 is configured to cut while being closed or while being opened, or both. A scissors-type cutter 92 configured to cut only while being closed may be used for blunt dissection, as is conventional.

A clamshell type of tissue separator tool 94 is shown in FIGS. 29-32. Tool 94 extends from tip 36 of slat 12. Tool 94 is shown in a closed state in FIGS. 29 and 30 and in a tissue separating, opened state in FIGS. 31 and 32. FIG. 33 shows a circular blade type of cutting tool 96 carried by and extending from tip 36 of slat 12. Tool 96 includes a blade 98 that can be rotated or reciprocated to cut tissue or other material.

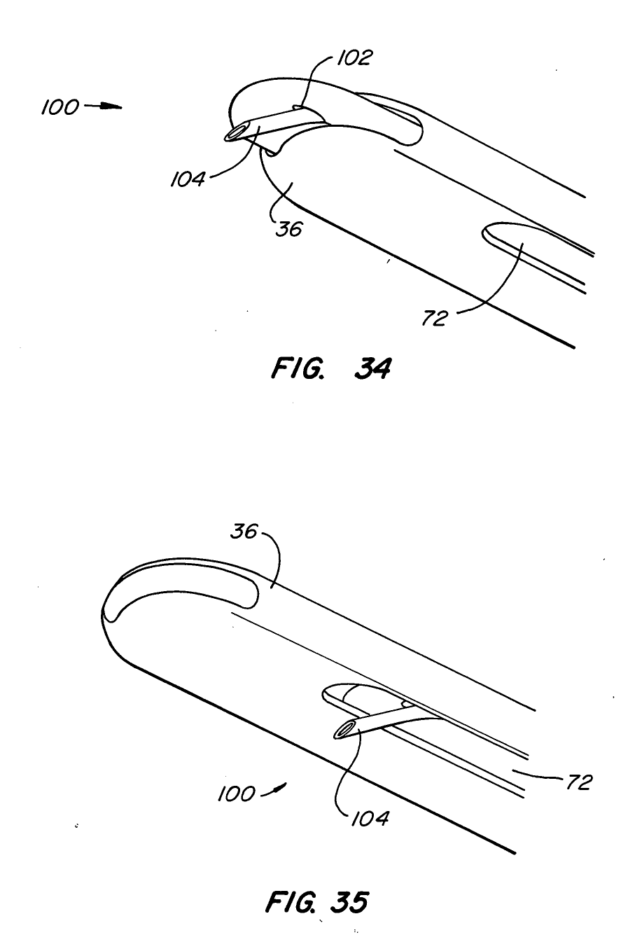

FIG. 34 shows a lumen re-entry tool 100. Tool 100, carried by and extending from tip 36 of slat 12, has an inwardly angled exit lumen 102 and a hollow needle 104 extending from the exit lumen. Tool 100 may be used to cross a vascular occlusion, such as described in U.S. Pat. No. 6,506,178. FIG. 35 shows re-entry tool 100 positioned along inner track 72 of slat 12 with hollow needle 104 passing through the inner track.

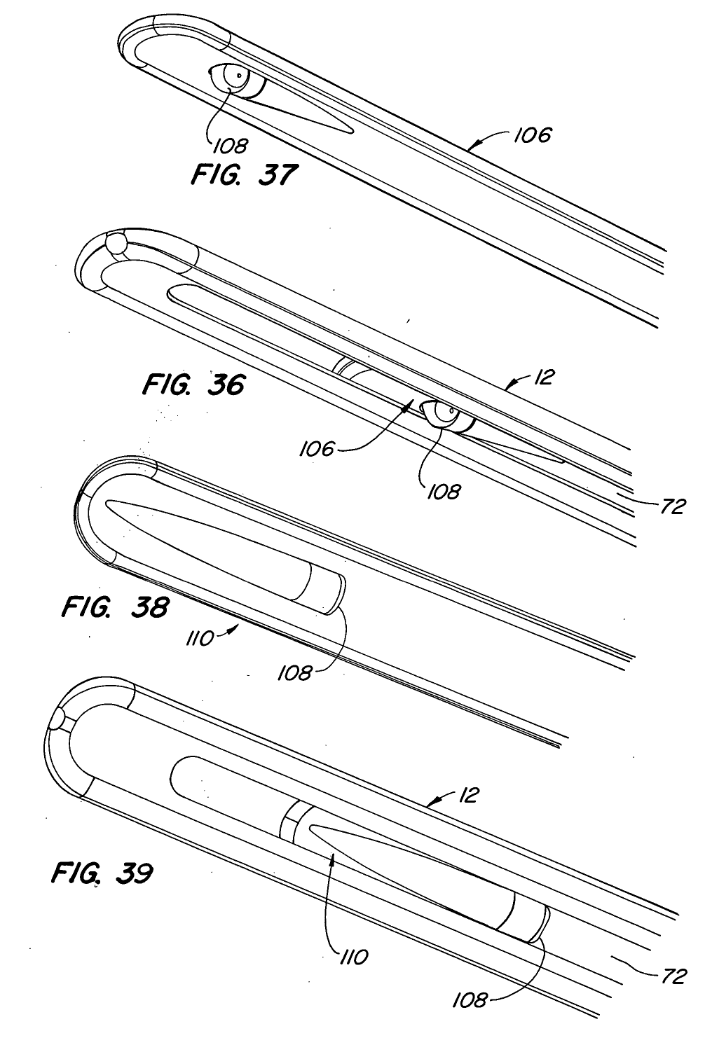

FIG. 36 illustrates a distally oriented cuplike cutter type of tool 106. Tool 106 is configured for insertion into inner track 72 of slat 12, as shown in FIG. 37. Tool 106 has a cuplike cutter 108 that can cut or shave material as it moves in a distal direction along inner track 72 of slat 12. Cutter 108 may be stationary or it may be rotated or reciprocated. The severed material may be aspirated away using suction. FIG. 38 illustrates a proximally oriented cuplike cutter type of tool 110. Tool 110 is also configured for insertion into inner track 72 of slat 12 as shown in FIG. 38. Tool 110 is moved proximally within slat 12 to engage and remove material.

FIGS. 40-46 disclose various tools that are mounted for movement along the inner track 72 of two adjacent slats 12. FIGS. 40 and 41 are overall and end views of a cutting tool 112 having first and second legs 114, 115 extending along inner tracks 72 of adjacent slats. Tool 112 also has a blade 116 secured to and extending between the distal ends of the leg. FIG. 42 shows a cutting tool 118 similar to that of FIG. 40 but having a notched blade 120. Cutting tool 122, shown in FIG. 43, is similar to that of FIG. 40 but has a rotating and/or reciprocating blade 124.

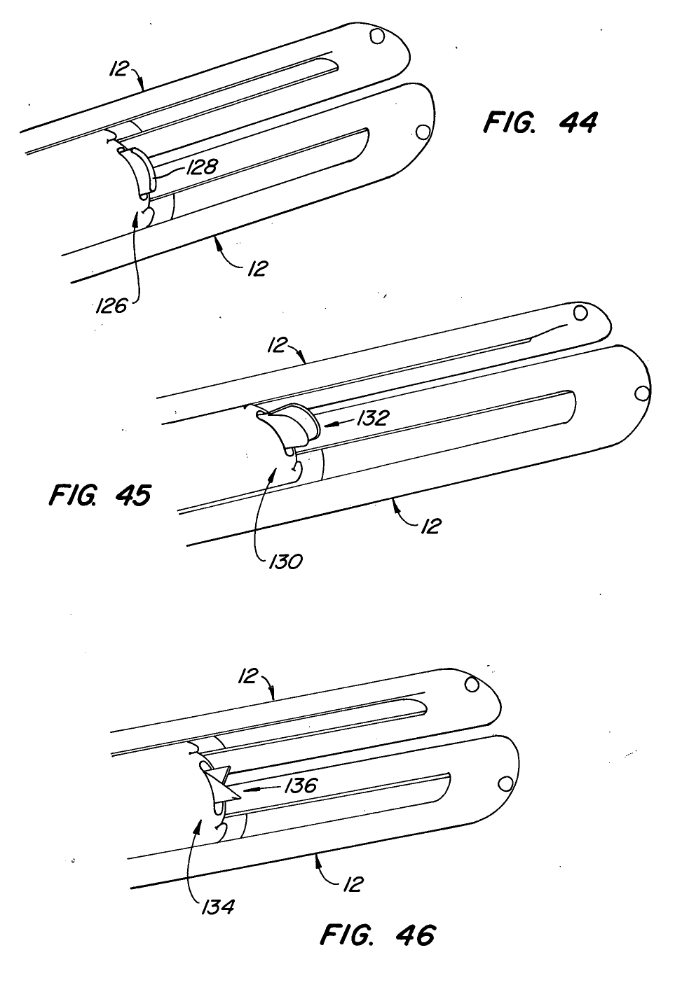

FIG. 44 shows a cutting tool 126 similar to that of FIG. 40 but having a chisel tip 128 instead of a blade. FIG. 45 shows a tool 130 similar to that of FIG. 40 but having a clamshell type of tissue separator 132 instead of a blade. Tissue separator 132 is similar to the distal end of clamshell tool 94 of FIGS. 29-32. FIG. 46 shows a cutting tool 134 similar to that of FIG. 40 but having a scissors type of cutter 136 instead of a stationary blade. Cutter 136 may, like scissors tool 92 of FIG. 28, be used to cut tissue or other material while being opened or while being closed, or both. A cutter 136 configured to cut only while being closed may be used for blunt dissection, as is conventional.

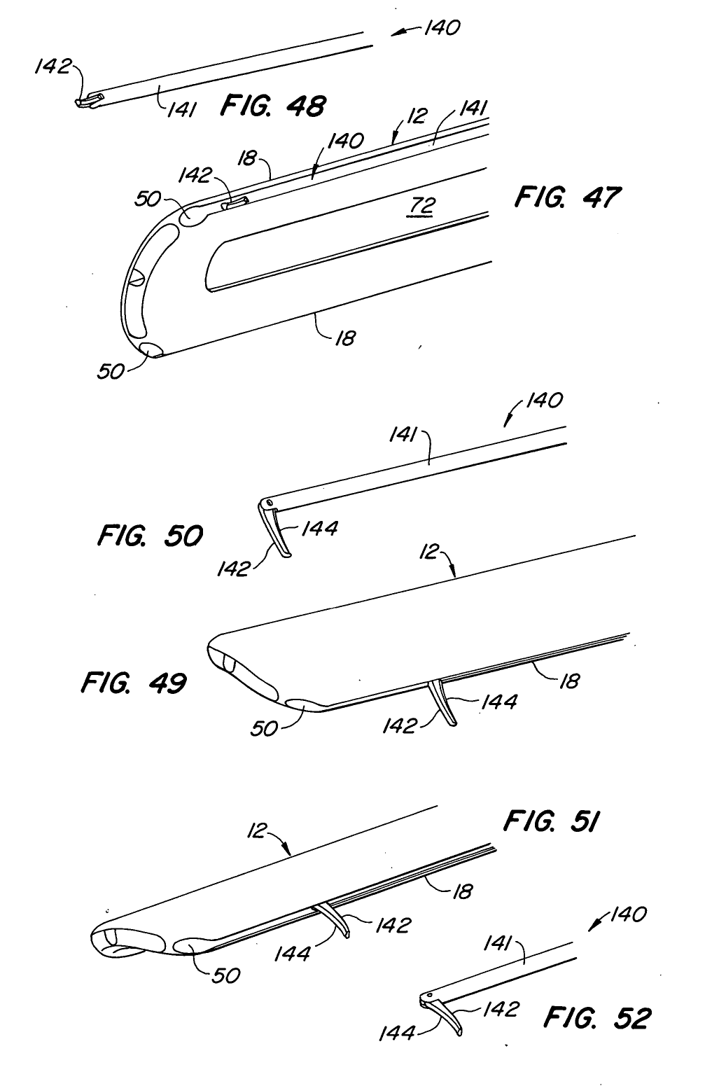

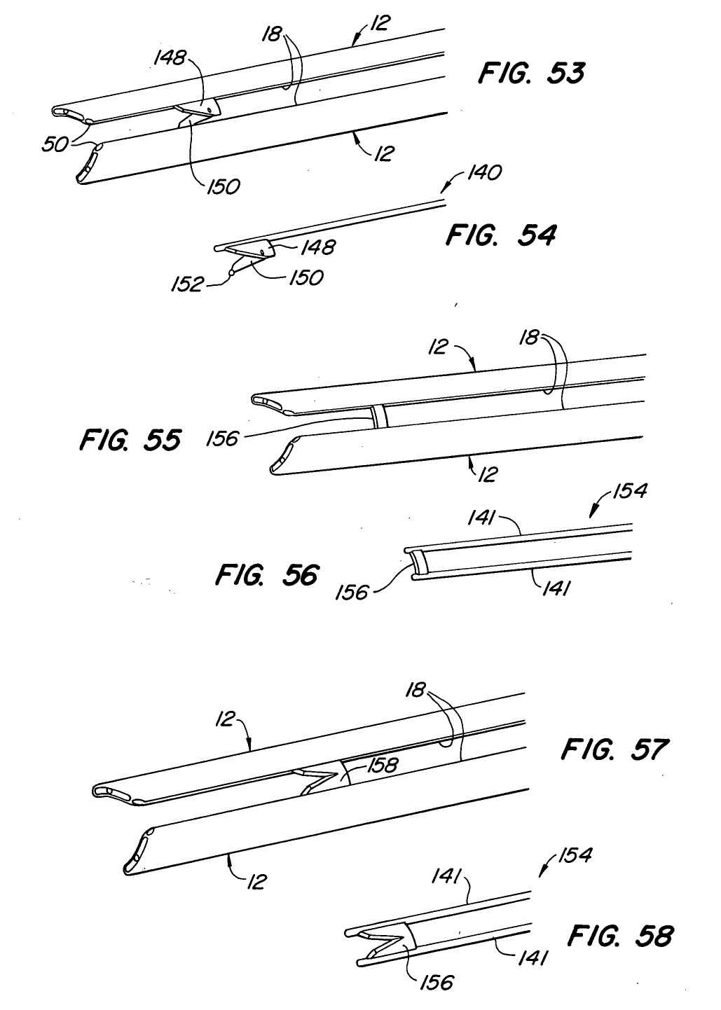

FIGS. 47 through 58 disclose various embodiments in which slats 12 have keyhole type of openings 50 along side edges 18 to permit an elongate cutter to be guided along the edge of one slat, see FIGS. 47-52, or between the side edges of adjacent slats, see FIGS. 53-58. FIGS. 47 and 48 disclose an elongate cutter 140 having an elongate member or shaft 141 with a blade 142 pivotally mounted at its distal end. Blade 142 is shown in its retracted state. FIGS. 49 and 50 disclose an embodiment similar to that of FIGS. 47 and 48 but with its blade 142 in its extended, use state. Blades 142 for both embodiments of FIGS. 47-50 have proximally facing cutting edges 144. FIGS. 51 and 52 illustrate an embodiment similar to that of FIGS. 49 and 50 but with a distally facing cutting edge 144.

FIGS. 53 and 54 show a further embodiment in which cutter 140 include a scissors type of blade assembly 146 at the distal end of shaft 141. Blade assembly 146 includes a fixed blade 148 fixed to and extending from shaft 141 and a moveable blade 150 pivotally mounted to blade 148. Both blades 145 and 150 have distally facing cutting edges. The tip of blade 150 has a ball 152 sized to slide within the opening 50 of an adjacent slat. This permits blade assembly 146 to sever tissue and other material between the adjacent edges 18 of slats 12 while permitting the distance between edges 18 to change. FIGS. 55 and 56 show a dual shaft elongate cutter 154 having two shafts 148 and a blade 156 extending between the distal ends of the shafts. With shafts 141 housed within openings 50, blade 142 extends between side edges 18 of adjacent slats 12. FIGS. 57 and 58 show an embodiment similar to FIGS. 55 and 56 but with a notched blade 158.

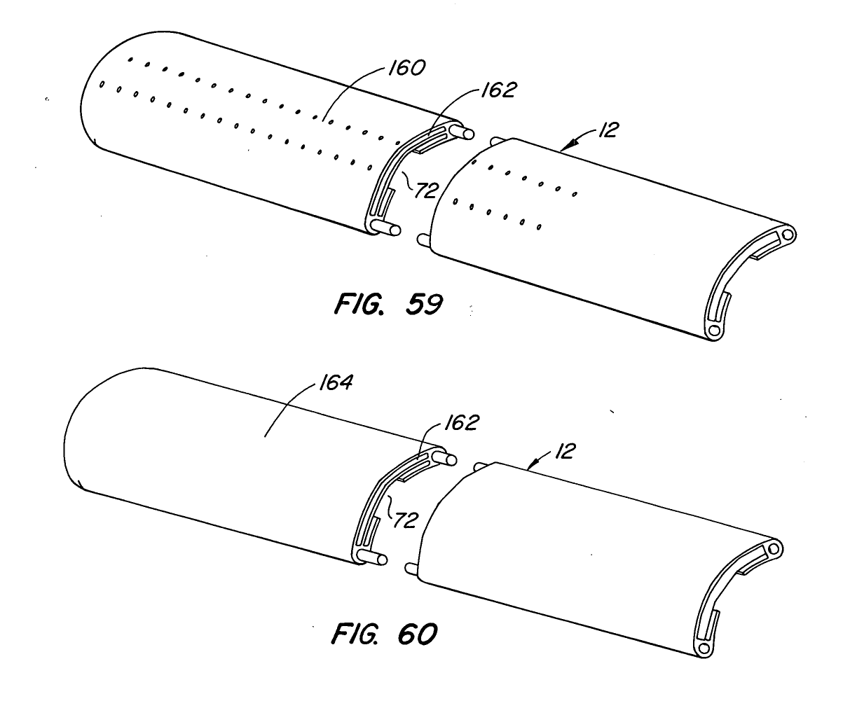

FIG. 59 shows slat 12 having a porous outer surface 160 opening into an interior passageway 162 of the slat for delivery of an agent through surface 160. For example, a therapeutic drug, diagnostic agent, gene therapy or other agent may be delivered to the vessel wall through porous outer surface 160. Porous outer surface 160 could, for example, include a permeable or semi permeable membrane instead of or in addition to discrete holes. FIG. 60 shows an embodiment similar to that of FIG. 59 but having a closed outer surface 164. Such a closed outer surface may be useful for radiation therapy or other treatments in which the agent is to remain in interior passageway 162.

An advantage of the invention is that slats 12 provide protection for the wall of blood vessel 15 from the various instruments, devices and tools used, typically along inner track 72. This permits the use of instruments and tools that would otherwise be considered too aggressive, and thus too dangerous, to use within a vessel because of the great potential to causing injury to the vessel. Therefore, one or more slats 12 may be used to dissect a cleavage plane 26 and also be used as a vehicle for the placement and use of a tool in a safe, effective manner.

Other modification and variation can be made to the disclosed embodiments without departing from the subject of the invention as defined in following claims. For example, the pusher guide track 48 for one or more of slats 12 may be used to help deliver a therapeutic agent to the vessel wall. Also, the various tools, including tools with cutting edges, maybe configured for the application of, for example, laser energy, RF energy, high-intensity focused ultrasound energy, vibration energy or heat energy, during use.

Any and all patents, patent applications and printed publications referred to above are incorporated by reference.

1. A vascular lining removal assembly comprising:
a plurality of slats, each having inner and outer surfaces, configured to pass along a vascular cleavage plane to removingly engage a length of vascular lining.

2. The assembly according to claim 1 wherein the slats have distal portions, said inner surfaces of said distal portions being concave and said external surfaces of said distal portions being convex.

3. The assembly according to claim 1 further comprising three of said slats.

4. The assembly according to claim 1 further comprising means for engaging the length of vascular lining to aid removal of the length of vascular lining.

5. The assembly according to claim 4 wherein the engaging means comprises means for constricting the length of vascular lining by the slats.

6. The assembly according to claim 5 wherein the slats comprise edges and the constricting means comprises at least one of:
a radially extendable tool carried by at least one slat movable between a first, radially retracted state and a second, radially extended state; and
slat coupling means for selectively joining adjacent edges of the slats.

7. The assembly according to claim 5 wherein the constricting means comprises a radially constrictable element placeable around the slats and movable between a first, relatively slat-unconstricting state and a second, slat inwardly-constricting state.

8. The assembly according to claim 7 wherein the radially constrictable element comprises at least one of:
a filament wound about the slats and movable between a first, relatively slat-unconstricting state and a second, slat inwardly-constricting state; and
a radially constrictable, generally tubular member surrounding the slats and movable between a first, relatively slat-unconstricting state and a second, slat inwardly-constricting state.

9. The assembly according to claim 4 wherein the engaging means comprises at least one of:
a one-way friction element configured to at least reduce slipping between the slats and the length of vascular lining during removal of the length of vascular lining;
an enhanced friction element at the inner surface of at least one of the slats, and a low friction shield removably covering the enhanced friction element to aid placement of the slats, the low friction shield of being removable after the slats have been passed along the cleavage plane and engage a length of vascular lining; and
means for selectively applying a suction force between at least one of the slats and the length of vascular lining to aid removal of the length of vascular lining.

10. The assembly according to claim 9 wherein the enhanced friction element comprises one or more of a one-way friction element and a high friction surface.

11. The assembly according to claim 1 further comprising a guide wire path along the length of each slat.

12. The assembly according to claim 1 wherein the slats comprise edges, wherein the slats comprise adjacent slats, and further comprising a tool carried by and extending between the adjacent slats, the adjacent slats comprising opposed edges.

13. The assembly according to claim 12 wherein the tool is slidably coupled to the opposed edges of the adjacent slats.

14. The assembly according to claim 12 wherein the tool comprises a cutter.

15. The assembly according to claim 12 wherein the tool comprises a tissue separator.

16. The assembly according to claim 12 wherein the tool comprises a lumen re-entry tool.

17. A vascular lining removal element comprising:
a slat, having inner and outer surfaces, configured to pass along a vascular cleavage plane to removingly engage a length of vascular lining; and
the slat having a distal portion having an average thickness T , an average width W and a length L, with W being at least 3 times T and L being at least 80 times W.

18. The element according to claim 17 wherein the target length of vascular lining to be removed is TL and L is 50% to 500% as long as TL.

19. The element according to claim 17 wherein the target length of vascular lining to be removed is TL and L is at least 150% as long as TL.

20. The element according to claim 17 wherein L is 400 mm long.

21. The element according to claim 17 wherein the slat has a generally constant width along its entire length.

22. A method for removing a length of vascular lining from a blood vessel comprising:
positioning a plurality of slats, each having inner and outer surfaces, along a cleavage plane of a blood vessel;
removingly engaging a length of vascular lining by the slats; and
removing the slats and a removed length of vascular lining therewith from the remainder of the blood vessel, the removed a length of vascular lining having a length RL.

23. The method according to claim 22 wherein the slats removing step comprises severing the removed length of vascular lining from the remainder of the blood vessel.

24. The method according to claim 22 further comprising:
using slats having:
distal portions at least 150% as long as RL; and
distal portions with an average thickness T, an average width W and a length L, with W being at least 3 times T and L being at least 80 times W.

25. The method according to claim 22 further comprising estimating the length of the length of vascular lining to be removed.

26. The method according to claim 22 further comprising using slats having distal portions, the distal portions having an average thickness T, an average width W and a length L, W being at least 3 times T, and L being at least 80 times W.

27. The method according to claim 26 wherein the length L is about 400 mm long.

28. The method according to claim 22 wherein the removingly engaging and removing steps are carried out without any user-applied constricting force on the slats.

29. The method according to claim 22 wherein the removingly engaging step comprises constricting the length of vascular lining by the slats.

30. The method according to claim 29 wherein the constricting step comprises manually grasping proximal ends of the slats and squeezing the slats against the length of vascular lining.

31. The method according to claim 29 wherein the constricting step comprises at least one of:
radially extending a tool carried by at least one slat between a first, radially retracted state and a second, radially extended state; and
joining adjacent edges of the slats.

32. The method according to claim 29 wherein the constricting step comprises placing a radially constrictable element around the slats after the slats positioning step and then radially constricting the radially constrictable element thereby radially constricting the slats against the length of vascular lining.

33. The method according to claim 22 wherein the removingly engaging step comprises at least one of:
applying a one-way friction element, positioned along at least one of the slats, to the length of vascular lining to at least reduce slipping between the slats and the length of vascular lining during the removing step;
removing, after the positioning step, a low friction shield from an enhanced friction element at an inner surface of at least one of the slats thereby exposing the enhanced friction element to the length of vascular lining, the low friction shield covering the enhanced friction element during the positioning step to aid placement of the slats, whereby exposing the enhanced friction element at least reduces slipping between the slats and the length of vascular lining during the first removing step; and
selectively applying a suction force between at least one of the slats and the length of vascular lining to aid removal of the length of vascular lining during the removing step.

34. The assembly according to claim 22 wherein the positioning step comprises guiding each slat using a guide wire.

35. A vascular lining removal assembly comprising:
a plurality of slats, each having inner and outer surfaces, configured to pass along a vascular cleavage plane to removingly engage a length of vascular lining; and
the inner surface of at least one of the slats comprising a passageway extending along at least a portion of the inner surface, at least a portion of the passageway opening inwardly to permit direct access to the length of vascular lining by a tool.

36. The assembly according to claim 35 further comprising a radially inwardly extendable tool carried by at least one slat, the tool being movable between a first, radially retracted state and a second, radially inwardly extended state so to engage the length of vascular lining in the second state.

37. The assembly according to claim 35 wherein the passageway extends along at least 50% of the inner surface.

38. The assembly according to claim 35 further comprising a tool engaging the passageway of at least one slat.

39. The assembly according to claim 38 wherein the tool comprises at least one of a cutter, a tissue separating tool and a lumen re-entry tool.

40. The assembly according to claim 38 wherein the tool engages and extends between the passageways of adjacent ones of the slats.

41. A vascular lining treatment assembly comprising:
a slat, having inner and outer surfaces, configured to pass along a vascular cleavage plane to engage a length of vascular lining;
the inner surface of the slat comprising a passageway extending along at least a portion of the inner surface, at least a portion of the passageway opening inwardly to permit direct access to the length of vascular lining; and
a tool engaging and passing along the passageway of the slat to permit engagement of the length of vascular lining by the tool.

42. A vascular lining removal element comprising:
a slat, having inner and outer surfaces, configured to pass along a vascular cleavage plane to removingly engage a length of vascular lining;
the inner surface comprising a passageway extending along at least a substantial portion of the inner surface, at least a portion of the passageway opening inwardly to permit direct access to the length of vascular lining by a tool; and
the slat having a distal portion having an average thickness T , an average width W and a length L, with W being at least 3 times T and L being at least 80 times W.

43. A method for removing a length of vascular lining from a blood vessel comprising:
positioning a plurality of slats, each having inner and outer surfaces, along a cleavage plane of a blood vessel, the inner surface comprising a tool passageway extending along at least a portion of the inner surface, at least a portion of the tool passageway opening inwardly to permit direct access to a length of vascular lining by a tool;
engaging the length of vascular lining through at least one tool passageway by a tool;
removingly engaging the length of vascular lining by the slats;
removing the length of vascular lining therewith from the remainder of the blood vessel; and
removing the tool from the blood vessel; and
removing the slats from the blood vessel.

44. The method according to claim 43 wherein the slats removing step comprises severing the length of vascular lining from the remainder of the blood vessel.

45. The method according to claim 43 wherein the length of vascular lining removing step and the tool removing step are carried out generally simultaneously.

46. The method according to claim 43 wherein the slats removing step is carried out after the length of vascular lining and tool removing steps.

47. The method according to claim 43 wherein the three removing steps are carried out generally simultaneously.
